(19) United States
(12) Patent Application Publication
(10) Pub. No.: US 2023/0136109 A1
(43) Pub. Date: May 4, 2023

(54) LIGHT-ACTIVATED PATHOGEN-KILLING MOLECULES AND USES THEREOF

(71) Applicant: EPIC VENTURES INC., Sidney (CA)

(72) Inventors: Heather BUCKLEY, Victoria (CA); Jeremy WULFF, Victoria (CA); Peter BERRANG, Sidney (CA); Mathieu LEPAGE, L'Huisserie (FR); Stefania MUSOLINO, Victoria (CA)

(21) Appl. No.: 17/908,200

(22) PCT Filed: Mar. 4, 2021
(86) PCT No.: PCT/CA2021/050290

§ 371 Date: Aug. 30, 2022

Related U.S. Application Data
(60) Provisional application No. 62/987,599, filed on Mar. 10, 2020.

(51) Int. Cl. A01N 59/00; C09D 5/14

(52) U.S. Cl. CPC A01N 59/00; C09D 5/14

(57) ABSTRACT

A series of novel molecular conjugates is disclosed herein, consisting of a photosensitizer moiety chemically bonded to at least one diazirine moiety. Such conjugates react in air and/or water with non-coherent visible light to form singlet oxygen, an energetic species known to be highly toxic to abroad range of pathogens, including bacteria, fungi, microbial biofilms and viruses. Such non-specific cellular damage limits the pathogen's ability to evolve into a resistant form. The diazirine moiety of the conjugate enables the reagent to be adhesively bonded to a plethora of substrates, such as, for example, polymers, non-woven fabrics, surgical masks and gloves, hospital gowns, hospital surfaces (such as counters, mattresses, curtains) medical implants, bandages, wound dressings, or air-filters. The resulting treated substrates act as an effective pathogen-killing barrier, providing protection against the spread of such pathogens to non-infected persons.

### FIELD

There is described light-activated pathogen-killing molecules that can be used to destroy bacteria, viruses and fungi that either adhere to or pass through a substrate.

### BACKGROUND

Historically, medical personnel have worn masks or sealed apparel to stop or minimize the passage of pathogens such as bacteria or viruses, but these precautions do not actively kill such pathogens. Non-medical personnel also frequently adopt the use of N95 masks during pandemics, but these provide only limited protection to viral threat.

Additionally, filter systems used in closed or partially-closed areas where air is re-circulated, such as, for example, in aircraft, window-sealed high-rise buildings, hospitals, submarines, etc. are generally not designed to actively kill pathogens, thereby allowing the spread of potentially dangerous pathogens via the air-circulating system.

Building upon the use of photosensitizing porphyrins in photodynamic therapy, Spontak and co-workers showed in 2018 that zinc tetra(4-N-methylpyridyl)porphyrin (ZnTMPyP4+) could be physically mixed into a polymer material to kill surface-associated bacteria and viruses. The mechanism of pathogen inactivation is believed to involve photochemical excitation of the zinc porphyrin with ambient light to produce an excited-state complex, followed by energy transfer to molecular oxygen present in the air. This affords singlet oxygen, which is known to be highly destructive to cells and viral capsids. Details are provided in: Peddinti, Scholle, Ghiladi and Spontak, Photodynamic Polymers as Comprehensive Anti-Infective Materials: Staying Ahead of a Growing Global Threat, ACS Appl. Mater. Interfaces 2018, 10, 25955-25959.

The utility of the above approach is limited, however, because the ZnTMPyP4+ powder cannot be adhesively attached to the polymer surface. As a result, it can be lost to the environment through abrasion or leaching. Moreover, the method of application (co-mixing of the molecule with the polymer substrate, followed by several rounds of heating, cooling, and grinding), would not be applicable to other materials, nor would it be applicable to pre-formed objects.

### SUMMARY

To address the limitations of the prior art, we have designed a novel diazirine-photosensitizer conjugate which can be attached to a variety of substrates, imbuing the resulting photodynamic substrate(s) with antibacterial, antiviral, and/or antifungal properties.

Such modified photodynamic substrates now become practical, in that these conjugates can be covalently bonded to myriad substrates or materials such as air-filter material, surgical masks and gloves, medical gowns, medical implants, bandages and wound dressings, medical instruments, walls, bed linens and surfaces, etc.

This new molecular conjugate, termed herein as a “Pathogen-Killing Molecular Adhesive” (PKMA), provides a pathogen-killing coating that can be adhesively fused to many kinds of substrates. Because singlet oxygen does not persist within the environment, the treated substrate is not expected to be hazardous to humans, but will effectively destroy bacteria, viruses and fungi that either adhere to the surface or pass through the substrate upon which the PKMA has been applied.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a molecular diagram of mono(diazirine) substituted porphyrins and corroles.

FIG. 2 is a molecular diagram of poly(diazirine) substituted porphyrins and corrroles.

FIG. 3 is a molecular diagram of poly(diazirine) substituted phthalocyanines.

FIG. 4 is sequence diagram of a method for the preparation of conjugates.

FIG. 5 is a schematic representation of a polymer cross-linking process.

FIG. 6 is a graphic representation of temperature ranges in which diazirine groups are thermally active.

FIG. 7 is a photographic representation of green crystals of zinc.

FIG. 8 is a graphic representation of quantified loss of viral activity

### DETAILED DESCRIPTION

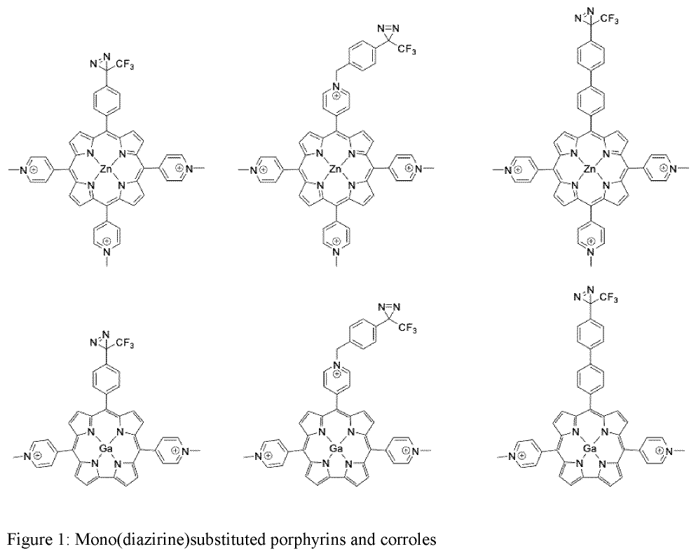

A Light-activated Pathogen-Killing Molecules and Uses Thereof will now be described with reference to FIG. 1 through FIG. 8.

As used herein, the term substrate includes, but is not limited to, polymers such as polyethylene, polypropylene and the like, materials such as non-woven fabrics produced from such polymers, finished products such as surgical masks and gloves, hospital gowns, mattresses, curtains and bed-linen, bandages, wound dressings, hospital surfaces (such as walls and counters) medical instruments and medical implants. It also includes products such as air-filters.

Disclosed herein are diazirine-photosensitizer conjugates comprising a photosensitizer moiety covalently bonded to at least one diazirine moiety.

Photosensitizers useful in the preparation of conjugates of the invention include, but are not limited to, porphyrins, corroles, phthalocyanines, phenothiazinium derivatives and the like. Such photosensitizers have the ability to absorb photons from applied or ambient light, and transfer some portion of the absorbed energy to other molecules. Among other available pathways, photosensitizers can excite O2 molecules, leading to the production of singlet oxygen or other highly reactive species like peroxide or superoxide.

Certain photodynamic molecules as disclosed herein can also be activated by an electric field, with or without the presence of light.

Other examples of photosensitizers useful in the preparation of conjugates of the invention include, but are not limited to, dyes such as rose bengal, eosin Y or methylene blue, and photoredox catalysts comprising coordination complexes of Ru or Ir, such as Ru(bipy)3 or Ir(ppy)3.

Examples of diazirines useful in the preparation of conjugates of the invention include, but are not limited to, trifluoromethyl diazirines, halodiazirines, alkoxy diazirines, alkyl and aryl diazirines as well as aryl ether diazirines.

Non-limiting examples of diazirine-photosensitizer conjugates of the invention include diazirine-porphyrins and diazirine-corroles having one diazirine moiety, as shown in FIG. 1.

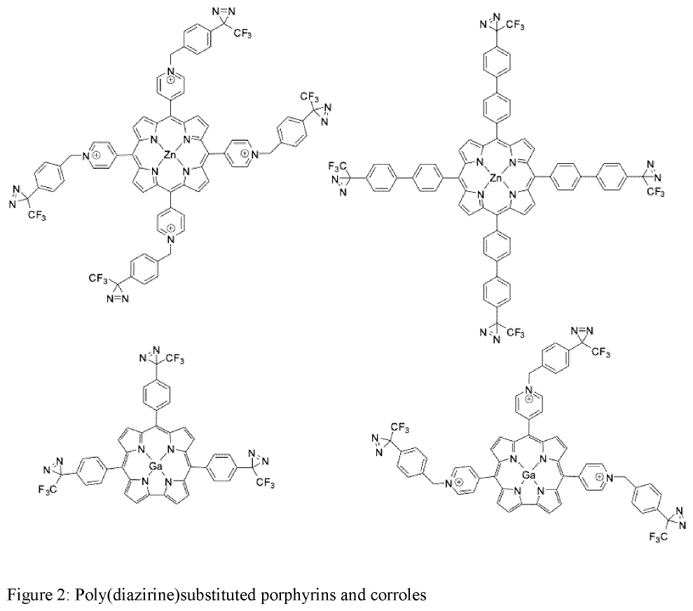

Further examples of conjugates of the invention include diazirine-porphyrins and diazirine-corroles having more than one diazirine moiety, as shown in FIG. 2.

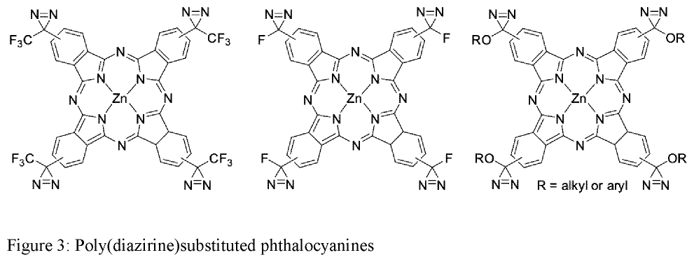

Other examples of conjugates of the invention include diazirine-phthalocyanines, as shown in FIG. 3.

A preferred conjugate of the invention is zinc 5,10,15,20-tetra(4-pyridyl)-21H,23H-porphine tetrakis(4-[3-(trifluoromethyl)-3H-diazirin-3-yl]benzobromide).

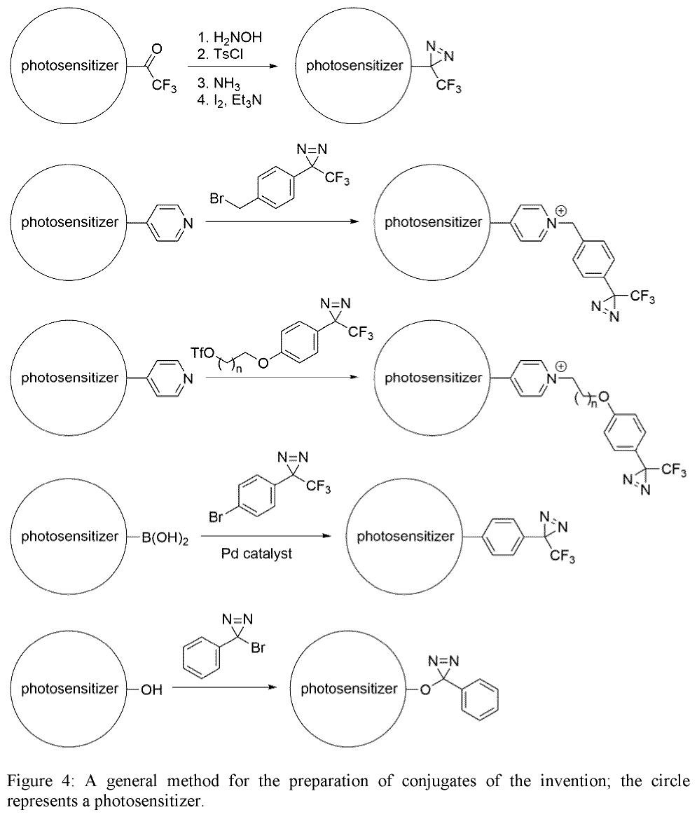

Conjugates of the invention may be prepared using techniques known in the art. For example, a precursor trifluoromethylketone may be first converted to the corresponding 0-tosyl oxime, then reacted with ammonia to produce a diaziridine intermediate, which can be oxidized to the desired diazirine. Alternatively, a pendant pyridyl group (or similar nucleophilic heterocycle) present on the photosensitizing molecule can be reacted with a suitable diazirine-containing electrophile, or else a pendant boronic acid group (or similar boron-containing functional group) present on the photosensitizing molecule can be reacted with a 3-(4-halophenyl)-3-(trifluoromethyl)-3H-diazirine and a suitable Pd catalyst, in a Suzuki cross-coupling. In still another method, a pendant hydroxyl group (or similar nucleophilic residue) on the photosensitizing molecule can be reacted with a suitable halodiazirine to effect conjugation. Illustrations of these non-limiting methods are shown in FIG. 4.

Conjugates may also be prepared using methods disclosed by Buckley et al. in Inorganic Chemistry 2014, 53 (15), 7941-7950. DOI: 10.1021/ic500714h.

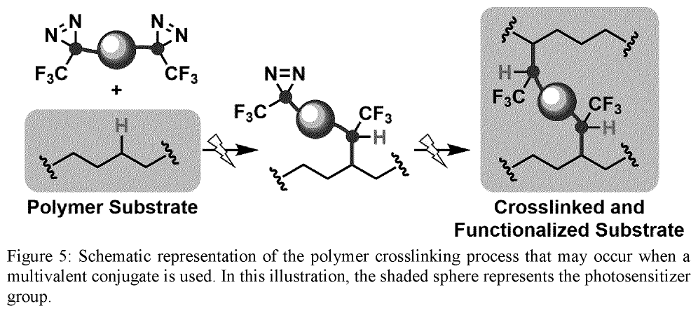

Without intending to limit the scope of the invention disclosed herein conjugates of the invention are designed to undergo loss of nitrogen upon thermal, photochemical, or electrical activation, resulting in carbene moieties that can undergo rapid C—H, O—H, or N—H insertion with substrate polymers, as shown in FIG. 5 (see Lepage, Simhadri, Liu, Takaffoli, Bi, Crawford, Milani and Wulff, A Broadly Applicable Cross-Linker for Aliphatic Polymers Containing C—H Bonds, Science 2019, 366, 875-878).

Conjugates having a single diazirine moiety provide a single point of attachment to a target substrate, such that the conjugate becomes covalently bound to the substrate, and is stable with respect to leaching. Examples of suitable substrates include, but are not limited to, polyethylene, polypropylene, cotton, and nylon.

Conjugates having more than one diazirine moiety may have multiple points of attachment to a target substrate (i.e. can act as cross-linkers), as shown in FIG. 5; the shaded blue sphere represents a photosensitizer. Conjugates with more than one moiety also provide superior loading efficiency in cases where the yield of the reaction with the substrate is less than 100%.

Conjugates of the invention may be adhesively fused to many kinds of substrates resulting in treated substrate(s) having anti-pathogenic properties such as antibacterial, antiviral, and/or anti-fungal properties.

Conjugates of the invention may be covalently fused to a broad range of finished products as well as to polymeric substrates such as air-filter material, surgical masks and gloves, medical gowns, medical implants, bandages and wound dressings, medical instruments, walls, bed linens and surfaces, etc.

For example, non-woven textiles such as polypropylene produced through a melt-blown process (MBPP) may be employed as substrates.

The conjugates disclosed herein may be termed “Pathogen-Killing Molecular Adhesives” (PKMA). They can be used to provide a coated substrate having a pathogen-killing coating that can be adhesively fused to many substrates. Because singlet oxygen does not persist within the environment, the treated substrate is not expected to be toxic to humans, but will effectively destroy bacteria, viruses and fungi that either adhere to the substrate or pass through the substrate upon which the PKMA has been applied.

The chemical process whereby the diazirine moiety can be activated to adhere to a substrate is disclosed in Provisional Patent Application No. 62/839,062 and in Lepage, Simhadri, Liu, Takaffoli, Bi, Crawford, Milani and Wulff, A Broadly Applicable Cross-Linker for Aliphatic Polymers Containing C—H Bonds, Science 2019, 366, 875-878, which disclosures are incorporated herein by reference.

The PKMA molecule may be dissolved in a suitable solvent to facilitate application to a substrate, for example pentane, diethyl ether, acetone, an alcohol such as methanol or ethanol, water, and super-critical CO2.

Substrates may then be soaked in this solution, and the solvent evaporated to provide an adsorbed layer of the PKMA. Activation of the diazirine moiety by heat, light, or electric potential results in adhesive bonding of the PKMA molecules to the substrate, and to itself.

Alternatively, a solution of the PKMA, or even neat PKMA in the absence of solvent, can be “painted” onto larger surfaces, such as hospital walls other surfaces. Once again, activation of the diazirine moiety (e.g. by light) creates a pathogen-killing surface.

### Example 1

Synthesis of zinc 5,10,15,20-tetra(4-pyridyl)-21H,23H-porphine tetrakis(4-[3-(trifluoromethyl)-3H-diazirin-3-yl]benzobromide). Zinc 5,10,15,20-tetra(4-pyridyl)-21H,23H-porphine (ZnTPyP) (70 mg, 0.102 mmol) was dissolved in 2 mL of DMF, then 4-[3-(trifluoromethyl)-3H-diazirin-3-yl]benzyl bromide (171 mg, 0.61 mmol) was added to the solution and the reaction was heated to 50° C. and stirred vigorously for 16 h. The solvent was then evaporated under reduced pressure and the crude product (211 mg, 0.10 mmol, 99%) was obtained as dark green solid. 1H NMR (300 MHz, MeOD) δ 9.48 (d, J=6.6 Hz, 8H), 9.14 (s, 8H), 8.94 (d, J=6.7 Hz, 8H), 7.99 (d, J=8.5 Hz, 8H), 7.54 (d, J=7.9 Hz, 8H), 6.29 (s, 8H). 13C NMR (126 MHz, MeOD) δ 164.80, 162.40, 161.73, 150.38, 144.27, 141.90, 139.40, 136.64, 134.68, 134.06, 131.76, 131.69, 130.87, 129.77, 129.72, 128.90, 128.36, 127.87, 127.71, 123.50 (q, J=273.9 Hz), 117.33, 64.76, 36.96, 35.43, 32.57, 31.64, 29.45 (q, J=40.7 Hz). 19F NMR (283 MHz, MeOD) δ −66.88.

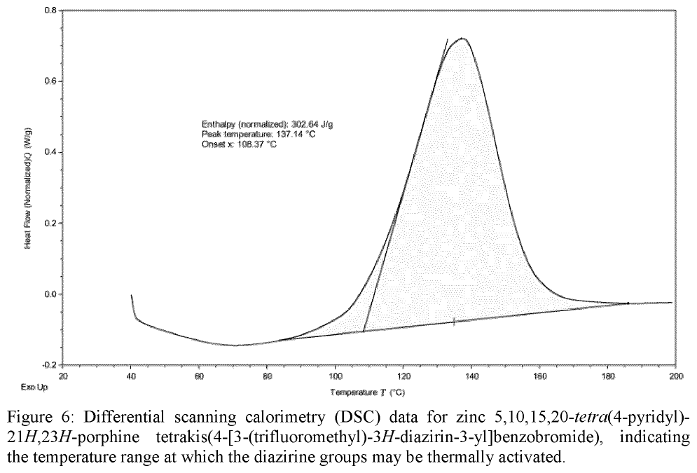

FIG. 6 shows the differential scanning calorimetry (DSC) data for zinc 5,10,15,20-tetra(4-pyridyl)-21H,23H-porphinetetrakis(4-[3-(trifluoromethyl)-3H-diazirin-3-yl]benzobromide), indicating the temperature range at which the diazirine groups may be thermally activated.

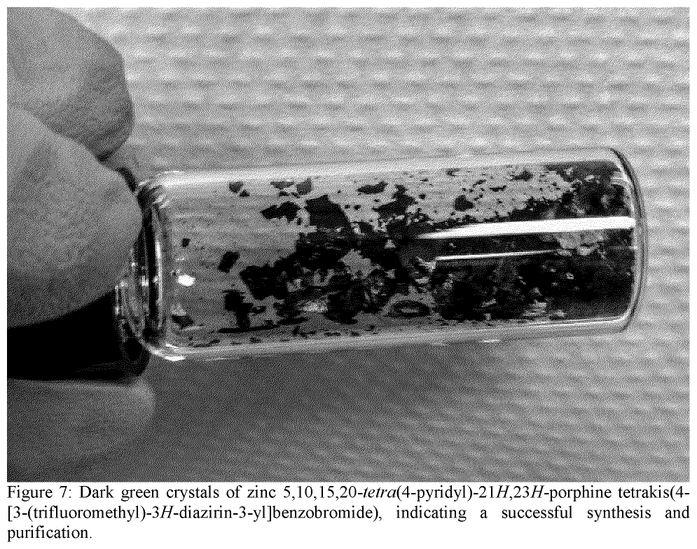

FIG. 7 shows green crystals of zinc 5,10,15,20-tetra(4-pyridyl)-21H,23H-porphine tetrakis(4-[3-(trifluoromethyl)-3H-diazirin-3-yl]benzobromide), indicating a successful synthesis and purification.

### Example 2—Anti-Viral Activity

Step 1: Functionalization of Melt-Blown Polypropylene (MBPP) with PKMA.

MBPP was cut into 4 cm diameter circles (white in colour) and placed in 4 cm diameter aluminum weigh boats. Each MBPP piece was submerged in a methanol solution of the compound of Example 1 (equating to a 1:10 weight ratio of PKMA:MBPP) and was subsequently covered with aluminum foil and allowed to incubate at room temperature for 1 hour. The aluminum pans were then uncovered and the methanol solvent was allowed to evaporate in the dark for 16 hours which resulted in a saturated green MBPP. The pans containing MBPP and evaporated PKMA were then incubated for 4 hours at 120° C. The resulting MBPP-PKMA textiles (5 pieces) were then washed with methanol to remove any residual non-bound PKMA by incubating the textile in 10 mL of methanol, changing the solution 10 times at different time intervals. The functionalized materials were then incubated in 500 mL of methanol for 60 hours, twice, to ensure there was no remaining free PKMA. PKMA was not observed by UV-Vis absorption of the last two incubation periods. The MBPP-PKMA textiles were then air dried and used for virus inactivation testing.

### Step 2: Virus Inactivation Testing

MBPP-PKMA was punched to precisely fit the bottom of a 96-well plate. MBPP and MBPP-PKMA were tested with 9 replicates and controls (empty wells) were completed in triplicate. 10 μL of Influenza A/California/07/09 stock solution was added to each well at a concentration of 5.98×10 7 platelet forming unit (PFU)/mL. The plate was exposed for 60 min to visible light (31,951−30,198 lux at the 96-well plate) with a temperature range between 24 (start) to 30.2° C. (end). Following exposure, 50 μL phosphate buffered saline (PBS) was added to remove remaining viruses from the MBPP-PKMA and pooled in triplicates, resulting in 3 samples each from MBPP-PKMA and MBPP, and 1 sample for negative control. The samples were split into aliquots and stored at −80° C. Viruses were then titered in 10-fold serial dilutions in Dulbecco's Modified Eagle Medium (DMEM) supplemented with 0.00075% Trypsin on ice. The concentration of in-vitro fertilization (IVF) was determined by plaque assay. MDCK cells that were previously seeded in a 6-well plate were inoculated with 200 μL of virus at each dilution in triplicate. After 1 hour adsorption with tilting every 15 minutes, inoculum was washed away and cells were overlaid with DMEM containing 1% Noble Agar and 0.0075% Trypsin. MDCK cells were stained with Neutral Red in the 6-well plate after infecting for 48 h. Antiviral efficacy was calculated by counting the number of remaining plaques.

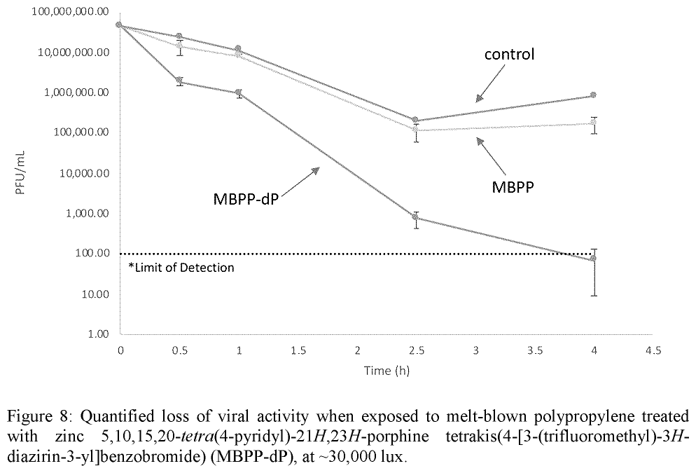

A plot of measured plaque forming units against time (FIG. 8) indicated that surface-bound PKMA inactivated the virus with a logarithmic relationship, reducing the concentration of viruses by 4 orders of magnitude (i.e. by 99.99%) in a four hour time period.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

The scope of the claims should not be limited by the illustrated embodiments set forth as examples, but should be given the broadest interpretation consistent with a purposive construction of the claims in view of the description as a whole.

What is claimed is:

1. A molecular conjugate comprising a photosensitizer moiety chemically bonded to at least one diazirine moiety.

2. The conjugate of claim 1 wherein the photosensitizer moiety is selected from the group consisting of a porphyrin, a corrole, a phthalocyanine and a phenothiazinium.

3. The conjugate of claim 1 wherein the diazirine moiety comprises a trifluoromethyl aryldiazirine group.

4. The conjugate of claim 3 wherein the photosensitizer is selected from the group consisting of a porphyrin, a corrole, a phthalocyanine and a phenothiazinium.

5. The conjugate of claim 4 wherein the conjugate is a monovalent compound as shown in FIG. 1.

6. The conjugate of claim 4 wherein the conjugate is a multivalent compound as shown in FIG. 2.

7. The conjugate of claim 4 wherein the conjugate is a compound as shown in FIG. 3.

8. The conjugate zinc 5,10,15,20-tetra(4-pyridyl)-21H,23H-porphine tetrakis(4-[3-(trifluoromethyl)-3H-diazirin-3-yl]benzobromide).

9. A method of preparing a conjugate of claim 1 as shown in FIG. 4.

10. A coated material comprising a substrate and a conjugate of claim 1.

11. The coated material of claim 10 wherein the conjugate is adhesively bonded to the substrate.

12. The coated material of claim 10 wherein the conjugate is covalently fused to the substrate.

13. The coated material of claim 12 wherein the substrate is a polymeric material.

14. The coated material of claim 13 wherein the substrate is a non-woven textile.

15. The coated material of claim 10 wherein the substrate is a finished product.

16. A method of preparing a coated material of claim 10 comprising the steps of dissolving a conjugate of claim 1 in a suitable solvent and applying the resulting solution to the material.

17. The method of claim 16 wherein the solvent is selected from the group consisting of pentane, diethyl ether, acetone, methanol, ethanol, water, and super-critical CO2.

18. A method of preparing a coated material of claim 10 by directly applying a conjugate of claim 1 to the material.

19. The use of a conjugate of claim 1 to provide a material having anti-pathogenic properties.

20. The use of a conjugate of claim 19 to provide a material having anti-viral properties.
